(19) United States
(12) Patent Application Publication
(10) Pub. No.: US 2023/0240978 A1
(43) Pub. Date: Aug. 3, 2023

(54) SYSTEMS AND METHODS FOR PRODUCING A NITRIC OXIDE BATH AND METHODS OF USE

(71) Applicants: C. Michael Miller, Anaconda, MT (US); David A. Bell, Farmington, UT (US); Allyson N. Miller, Anaconda, MT (US)

(72) Inventors: C. Michael Miller, Anaconda, MT (US); David A. Bell, Farmington, UT (US); Allyson N. Miller, Anaconda, MT (US)

(21) Appl. No.: 18/101,820

(22) Filed: Jan. 26, 2023

Related U.S. Application Data
(63) Continuation-in-part of application No. 17/814,126, filed on Jul. 21, 2022.
(60) Provisional application No. 63/224,533, filed on Jul. 22, 2021.
(60) Provisional application No. 63/349,458, filed on Jun. 6, 2022.

(51) Int. Cl. A61K 9/00; A61K 9/12

(52) U.S. Cl. CPC A61K 9/0014; A61K 9/12; A61K 31/185

(57) ABSTRACT

Systems, products, compounds, and methods can be applied and/or used by a person to facilitate and promote the production of nitric oxide to be used by a person for health benefits. The systems, products, and devices include a nitric oxide bath system. The systems and methods can comprise one or more compounds that when mixed are adapted to produce nitric oxide gas, with at least one nitrite compound and at least one acid compound. The nitric oxide gas may then be delivered to a user transdermally via one or more bath applications.

### RELATED APPLICATIONS

This patent application is a continuation-in-part of U.S. patent application Ser. No. 17/814,126, filed Jul. 21, 2022, which claims the benefit of U.S. Provisional Patent Application Ser. No. 63/224,533, filed Jul. 22, 2021; and this patent application claims the benefit of U.S. Provisional Patent Application Ser. No. 63/349,458, filed Jun. 6, 2022; all of which are hereby incorporated by reference herein in their entireties. Any and all applications for which a foreign or domestic priority claim is identified in the Application Data Sheet of the present application are hereby incorporated by reference herein under 37 CFR 1.57.

### BACKGROUND

### Field of the Invention

This invention relates to apparatus and methods for delivery of nitric oxide gas, and more specifically for devices and configurations for time-release granules, tablet shapes, and methods of use for nitric oxide baths, including foot baths.

### Description of the Related Art

There are numerous benefits documented regarding the use of nitric oxide to help or treat humans having a variety of ailments, diseases, illnesses, or afflictions. The discovery of certain nitric oxide effects in live tissue garnered a Nobel Prize. Much of the work in determining the mechanisms for implementing, and the effects of, nitric oxide administration are reported in literature.

Nitric oxide is considered an unstable molecule. This makes it difficult to develop a product or method that can consistently promote the formation of, or provide usable, nitric oxide to a person.

### SUMMARY OF THE INVENTION

What is needed is a product and method that promotes the use of nitric oxide by a person by means of a foot bath, or similar process.

In accordance with the foregoing, certain embodiments of a product, compound and method in accordance with the invention provide a topical product that can be applied or used by a person to facilitate and promote the production of nitric oxide to be used by a person, especially with respect to the treatment of various maladies, illnesses, biofilms, injuries, low blood circulation, or the like. The topical product may be suitably formulated to promote production of nitric oxide for use in human cells and tissues.

In one embodiment, one or two tablets are purposefully shaped in a way to regulate production of nitric oxide. A tablet shape may be purposefully selected to have a desired surface area to volume ratio. For example, a tablet shaped like a disc, with a higher surface area to volume ratio, may allow the tablet to dissolve relatively quickly in a solution. Whereas, a tablet shaped like sphere, with a relatively lower surface area to volume ratio, may allow the tablet to dissolve relatively slower in a solution.

In one embodiment, a product includes granules of reactants that are coated to allow for a time-release and sustained production of nitric oxide in a water bath. The thickness of such a coating may also be varied.

According to some embodiments, the present disclosure relates generally to a system for using one or more mediums to produce nitric oxide in a bath, and more specifically, a system for producing nitric oxide in a water bath for promoting transdermal absorption of nitric oxide to provide nitric oxide therapy.

Nitric oxide, or nitrogen monoxide, is a colorless gas and has the molecular formula NO. In mammals, including humans, nitric oxide is considered a “signaling molecule” in many physiological processes. It is a key vertebrate biological messenger, playing a role in a variety of biological processes. It acts as a messenger molecule, transmitting signals to cells in the cardiovascular, nervous, and immune systems. Nitric oxide is a free radical. The nitric oxide molecule's possession of a free radical makes it much more reactive than other signaling molecules. Nitric oxide's small molecular size enables it to diffuse through cell membranes and walls to perform a range of signaling functions in various bodily systems.

It is biosynthesized endogenously from oxygen, L-arginine, and NAPH (nicotinamide adenine dinucleotide phosphate) by various nitric oxide synthase (NOS) enzymes. The body synthesizes nitric oxide from the amino acid L-arginine by means of the enzyme NOS. Nitric oxide is formed by the oxidation of nitrogen. Reduction of inorganic nitrate may also produce nitric oxide.

Nitric oxide is an endothelium-derived relaxing factor. The endothelium, or inner liner, of blood vessels uses nitric oxide to signal the surrounding smooth muscle to relax, which results in vasodilation and increased blood flow. Nitric oxide's role in dilating blood vessels makes it an important controller of blood pressure. Nitric oxide is also produced by neurons and is used by the nervous system as a neurotransmitter to regulate functions ranging from digestion to blood flow to memory and vision. In the immune system, nitric oxide is produced by macrophages, which are a type of leukocyte that engulfs bacteria and other foreign particles that have invaded the body. The nitric oxide released by macrophages kills bacteria, other parasites, and tumor cells by disrupting their metabolism.

As people age, their ability to naturally produce nitric oxide diminishes. By the age of 50, a person's natural ability to produce nitric oxide may decrease by approximately 50%. What is needed is a simple and efficient system for supplementing nitric oxide availability for a person's natural physiological processes, including wound healing.

In one embodiment, two solutions or two separate mediums are used to initiate the production of nitric oxide (NO). A first solution may be a nitrite solution. A second solution may be an acidic solution. The first and second solutions may be placed into a water bath simultaneously or separately to initiate the production of nitric oxide.

In one embodiment, a first medium may include a nitrite and a second medium may include an acid. The first and second mediums may be packaged, or made available, in a single product that is composed to release the first and second mediums after the product is placed in a water bath.

According to certain aspects of the disclosure, what is needed are systems, products, apparatus, and methods of application that are easy to use, portable, reusable, and or/disposable that can also promote the use of nitric oxide by a person by means of a bath, such as a foot bath, a hand bath, or a similar process. Some embodiments of products, compounds, systems, and methods in accordance with this disclosure provide a nitric oxide gas product and systems adapted for a foot bath that can be applied or used by a person, especially with respect to the treatment of various maladies, illnesses, biofilms, injuries, or the like. The product and systems may be suitably formulated to promote production of nitric oxide for use in human cells and tissues.

According to some embodiments, systems, methods, products, and/or devices, a user may topically apply or use a product to facilitate and promote the production of nitric oxide to be absorbed by the user's skin. The user may topically apply such product for the treatments of various maladies, illnesses, biofilms, injuries, low blood circulation, etc. The topical product may be suitably formulated to promote production of nitric oxide for use in human cells and tissues. The product may be designed in a tablet shape that may be purposefully selected to have a desired surface area to volume ration. According to some embodiments, the tablet shape may include a disc with a higher surface area to volume ratio, which may allow the tablet to dissolve relatively quickly in a solution. According to some embodiments, the tablet shape may include a sphere with a relatively lower surface area to volume ratio, which may allow the tablet to dissolve relatively slower in a solution. According to some embodiments, the tablet may include a plurality of reactants. The tablet of reactants may be placed in a bath of water or soak, which may initiate a reaction with the reactants of the tablet and create an effervescent effect. According to some embodiments, the tablet of reactants may include granules of reactants that may be coated to allow for a time-release and/or sustained production of nitric oxide in a water bath. The thickness of the coating may be varied.

For purposes of this summary, certain aspects, advantages, and novel features of the invention are described herein. It is to be understood that not necessarily all such advantages may be achieved in accordance with the particular embodiments of the invention. Thus, for example, those skilled in the art will recognize that the invention may be embodied or carried out in a manner that achieves one advantage or group of advantages as taught herein without necessarily achieving other advantages as may be taught or suggested herein.

All of these embodiments are intended to be within the scope of the invention herein disclosed. These and other embodiments will become readily apparent to those skilled in the art from the following detailed description having reference to the attached figures and appendices, the invention not being limited to any particular disclosed embodiment(s).

According to certain embodiments, systems, methods, products, aspects, and/or applications a nitric oxide bath delivery system comprises a nitrite component; an acidic component; and a bath container adapted and configured to hold the nitrite component, the acidic component, and a bath medium during use. The bath medium can be water, saline, and/or a suitable solution in some embodiments. In some embodiments, at least one of the nitrite component and the acidic component comprises a solid portion that is configured and adapted to dissolve in the bath medium and provide effervescence to the bath during use. When combined in the bath, the nitrite component, the acidic component, and the bath medium are preferably adapted to be mixed to safely generate a therapeutically effective treatment dose of nitric oxide gas to an appendage of a user submerged in the bath during the gas generation period. At least one of the nitrite component and the acidic component is preferably adapted and configured to control a rate of generation of nitric oxide in the bath to provide an extended release gas generation period.

According to certain embodiments, systems, methods, products, aspects, and/or applications the bath container comprises a body portion that has a vented housing adapted and configured to receive at least one of a nitrite component and an acidic component. In certain embodiments, the nitrite component comprises one or more of potassium nitrite and sodium nitrite. In certain embodiments, the acidic component comprises one or more of hydrochloric acid, ascorbic acid, salicylic, and citric acid. Any suitable acid may be used. In certain embodiments, a sodium bicarbonate component is adapted and configured to be in the bath during the gas generation period. In certain embodiments, a fragrance component is adapted and configured to be in the bath during the gas generation period. In certain embodiments, a portion of at least one of the nitrite component and the acidic component is formed as a tablet. In certain embodiments, a tablet comprises at least one or more of an extended time release shape, configuration, composition, and/or coating. In certain embodiments, a portion of at least one of the nitrite component and the acidic component is positioned within a dissolvable container and/or pouch adapted and configured to at least partially dissolve within the bath to initiate the gas generation period. In certain embodiments, at least one of the nitrite component and the acidic component comprises a liquid portion that is configured and adapted to form a solution with the bath medium during the gas generation period. In certain embodiments, a surfactant and/or foaming component is configured and adapted to form a foam and/or bubbles on a layer of the bath medium during the gas generation period such that the foam and/or bubble layer forms a barrier that substantially hinders nitric oxide gas from escaping out of the bath and substantially acts to improve the transdermal absorption of nitric oxide gas into an appendage of the user positioned within the bath below the barrier during the gas generation period.

According to certain embodiments, systems, methods, products, aspects, and/or applications a method for delivery of nitric oxide treatment in a bath comprises providing a nitrite component; providing an acidic component; providing a bath container adapted and configured to hold the nitrite component, the acidic component, and a bath medium during use; and providing a bath medium. According to some embodiments, the bath medium is one or more of water, saline, and/or a suitable solution. The method can comprise positioning at least one of the nitrite component and the acidic component within the bath medium. The method can comprise at least one of the nitrite component and the acidic component comprises a solid portion that is configured and adapted to dissolve in the bath medium and provide effervescence to the bath. The method can comprise mixing at least the nitrite component and the acidic component within the bath medium, wherein when combined in the bath, the nitrite component, the acidic component, and the bath medium are adapted to safely generate a therapeutically effective treatment dose of nitric oxide gas to an appendage of a user submerged in the bath during a gas generation period. The method can comprise at least one of the nitrite component and the acidic component being adapted and configured to control a rate of generation of nitric oxide in the bath to provide an extended release gas generation period.

According to certain embodiments, systems, methods, products, aspects, and/or applications a method can include that the bath container with a body portion that has a vented housing adapted and configured to receive at least one of a nitrite component and an acidic component. The method can include positioning the at least one of a nitrite component and an acidic component within the vented housing. The method can include that the nitrite component comprises one or more of potassium nitrite and sodium nitrite. In some embodiments, the method can include that the acidic component comprises one or more of hydrochloric acid and citric acid. In some embodiments, the method can include providing a sodium bicarbonate component within the bath medium during the gas generation period. In some embodiments, the method can include providing a fragrance component within the bath medium during the gas generation period. In some embodiments, the method can include that a portion of at least one of the nitrite component and the acidic component is formed as a tablet, wherein the tablet comprises at least one or more of an extended time release shape, configuration, composition, and/or coating. In some embodiments, the method can include that a portion of at least one of the nitrite component and the acidic component is positioned within a dissolvable container and/or pouch adapted and configured to at least partially dissolve within the bath to initiate the gas generation period. In some embodiments, the method can include that at least one of the nitrite component and the acidic component comprises a liquid portion that is configured and adapted to form a solution with the bath medium during the gas generation period. In some embodiments, the method can include a surfactant and/or foaming component configured and adapted to form a foam and/or bubbles on a layer of the bath medium during the gas generation period such that the foam and/or bubble layer forms a barrier that substantially hinders nitric oxide gas from escaping out of the bath and substantially acts to improve the transdermal absorption of nitric oxide gas into an appendage of the user positioned within the bath below the barrier during the gas generation period.

### BRIEF DESCRIPTION OF THE DRAWINGS

The foregoing and other objects and features of the present invention will become more fully apparent from the following description and appended claims, taken in conjunction with the accompanying drawings, data, and/or charts. Understanding that these depict only typical embodiments of the invention and are, therefore, not to be considered limiting of its scope. Certain features and embodiments of this disclosure will now be discussed in detail with reference to the following figures. These figures are provided for illustrative purposes only, and the embodiments are not limited to the subject matter illustrated in the figures.

FIG. 1 illustrates an example of a hand press for forming a tablet according to certain embodiments.

FIG. 2 illustrates a schematic of a boundary layer around a solid dissolving into a solvent.

FIG. 3 illustrates a schematic of testing data reflecting concentrations of nitric oxide gas and nitrogen dioxide gas over time relative to first and second tablets.

FIG. 4 illustrates a schematic of testing data reflecting concentrations of nitric oxide gas over time relative to first and second tablets.

FIG. 5 illustrates an example of a pod according to certain embodiments.

FIG. 6 illustrates a schematic of testing data reflecting the release of nitric oxide relative to pods according to certain embodiments.

FIG. 7 illustrates a schematic of testing data reflecting the total and maximum release of nitric oxide relative to tablets.

### DETAILED DESCRIPTION OF SOME PREFERRED EMBODIMENTS

Systems, products, compounds, and methods can be applied and/or used by a person to facilitate and promote the production of nitric oxide to be used by a person for health benefits. The systems, products, and devices include a nitric oxide bath delivery system. The system can comprise one or more compounds that when mixed are adapted to produce nitric oxide gas, at least one nitrite compound and at least one acid compound. The compounds can be mixed and produce nitric oxide gas. The nitric oxide gas may then be delivered to a user via one or more bath applications.

The following detailed description is directed to certain specific embodiments. The invention(s) disclosed herein, however, can be embodied in a multitude of different ways as defined and covered by the claims. In this description, reference is made to the drawings, wherein like parts are designated with like numerals throughout. The features, aspects and advantages of the present invention will now be described with reference to the drawings of several embodiments that are intended to be within the scope of the development herein disclosed. These and other embodiments will become readily apparent to those skilled in the art from the following detailed description of the embodiments having reference to the attached figures, the invention not being limited to any particular embodiment(s) herein disclosed.

It will be readily understood that the components of the present invention, as generally described herein, could be arranged, and designed in a wide variety of different configurations or formulations. Thus, the following more detailed description of the embodiments of the systems, products, apparatus, and methods of the present invention, are not intended to limit the scope of the invention as claimed but are merely representative of various embodiments of the invention.

A person may obtain the benefits of nitric oxide therapy by utilizing a device designed to allow transdermal absorption of nitric oxide, such as a bath or soak. For example, a foot bath may be used to treat a person's feet with nitric oxide. Such treatment can help the skin of the feet, including any wounds or topical irregularities on a person's feet. Such treatment can also help the blood flow or circulation of the feet.

In one embodiment, a foot bath may include a tub that holds a suitable amount of water so that a person's feet can be immersed in the water. Then a tablet of reactants, or two tablets of separate reactants, may be dropped into the water initiating a reaction that produces nitric oxide gas. The tablet, or one or both of the tablets, of reactants may be effervescent. Sodium bicarbonate, or baking soda, may be utilized to help promote or cause the effervescence. Other components may be included in the tablets, such as Epsom salts, fragrances, or the like.

Generally, the reactants will be comprised of a nitrite compound or medium and an acidified, or acidic, compound or medium. The nitrite compound or medium may be formulated and formed in any suitable manner and the concentration of reactants can be adjusted as desired as long as the intended reaction and sufficient concentrations of nitric oxide is obtained. The acidified compound or medium may be formulated in any suitable manner and the concentration of the reactants can be adjusted as desired as long as the intended reaction and sufficient concentrations of nitric oxide are obtained.

In one embodiment, a first compound, or nitrite compound, may include sodium or potassium nitrite. A second compound, or acidified compound, may include ascorbic acid or citric acid.

The use of ascorbic acid as the acidified compound or medium has been found to produce less nitrogen dioxide, or even no nitrogen dioxide, thus promoting a more pure production of nitric oxide gas.

Nitric oxide gas may have an “affinity” to capillary beds, which results in the nitric oxide gas virtually seeking out, or being attracted to, capillaries and capillary beds to be absorbed into them.

In one embodiment, a layer of foam may be formed on the top of the foot bath. Such a layer of foam may help to trap nitric oxide gas that can escape the water of the foot bath before it is absorbed by a user.

In one embodiment, a bath or soak may be engineered so that a bottom compartment is structurally separated from a space where a user's feet would be. The bottom compartment, or lower containment, may be where the reactants are used to initiate the production of nitric oxide gas and a foam, where the foam includes bubbles that contain the nitric oxide gas, and the foam fills up the space where a user's feet would be. Such an embodiment could be described as a foam bath or soak.

In one embodiment, the shape of the reactant tablet, or separate reactant tablets, is formed in a manner to have a certain, desired surface area to volume ratio. For example, a reactant tablet could be formed in the shape of a disc, a sphere, a cube, a cylinder, or any desired shape. A tablet of any shape may be comprised of any desired reactants, sodium bicarbonate, fragrance materials, or other desired tablet components.

In one embodiment, the reactant tablets or pellets may be granular and coated in a manner that allows the granules to have a time-release function. For example, reactant tablets may be coated with polyethylene glycol, or any suitable coating, so that the coating must substantially dissolve before the reactants come in contact with a solution that can activate the respective reactants. A suitable coating material should not include anything that may react with the reactants being coated. Also, one coating material may be suitable for nitrite reactants while a separate coating material may be suitable for acidified reactants.

The thickness of the coating on the reactants can vary so that the time to dissolve the coating varies. The coated reactants with varying thicknesses may be utilized to prolong and/or maintain nitric oxide gas production. Moreover, the coated reactants with varying thicknesses may be portioned so that more nitric oxide gas is produced at a desired time interval as compared to other time intervals.

The present invention may be embodied in other specific forms and combinations without departing from its spirit or essential characteristics. The described embodiments are to be considered in all respects only as illustrative, and not restrictive. The scope of the invention is, therefore, indicated by the appended claims, rather than by the foregoing description. All changes which come within the meaning and range of equivalency of the claims are to be embraced within their scope.

According to some embodiments, a method for producing nitric oxide in a bath comprises providing a tub. The method can include filling the tub with water to substantially cover a portion of a user's skin. The method can include providing a tablet comprising nitrite reactants and acidified reactants, wherein the acidified reactants consist of ascorbic acid. The method can include placing the tablet in the water. The method can include producing nitric oxide via the tablet. The method can include absorbing, transdermally, at least a portion of the nitric oxide gas through at least a portion of the user's skin. The method can include providing a first tablet that comprises nitrite reactants and providing a second tablet that comprises acidified reactants. The method can include a tablet that is coated so as to time the initiation of the production of the nitric oxide gas.

According to certain embodiments, a method for producing nitric oxide gas in a manner that can be utilized by a user in a bath or a soak. The reactants used to produce nitric oxide gas may be coated pellets that allow for timed release of the reactants, thereby controlling and maintaining the production of nitric oxide gas in the bath or soak. According to some embodiments, nitric oxide time-release granules can be used.

Given the success of a nitric oxide gas/foam two-part application on wounds, acne, and the like, it is desired to develop a tablet of a nitrite salt, and organic acid, and other ingredients to add to a foot bath for the care of wounds, neuropathy, and ulcers. There are several versions of such a tablet possible with many variables or desired attributes of the bath that have been specified.

The primary action of creating NO gas is a well understood attribute according to some embodiments. The tablet will contain sodium nitrite to create the nitrite ion when the salt dissolves in water. The tablet will also contain citric acid or ascorbic acid to provide the acidic proton to react with the nitrite ion to create NO gas. Note that if ascorbic acid is used, the coproduction of NO2 gas will be minimized.

The creation of NO gas will make the tablet dissolve in an effervescent manner. It may be desirable to create more gas bubbles. It is possible that more gas bubbles will be preferable for better mixing of the ingredients in the bath, a better treatment experience for the user, or a more pleasing experience overall. If a more vigorous effervescent action is required or desired, a simple addition of baking soda (sodium bicarbonate) to the tablet ingredients will accomplish the goal. Note, it may be necessary to add a commensurate amount of citric acid or ascorbic acid to the ingredients to offset the baking soda neutralization.

The required robustness of the tablet to shipping and other handling can be determined. The tablet could be formulated with a binding agent specified for the pressure and time spent under pressure in the press, or the tablet could be packaged in such a way as to protect it possibly with shrink wrap.

The shape of the tablet can affect the effervescent tablet application in two ways. The shape of the tablet will control the rate at which the tablet dissolves in water. Therefore, the shape will control the rate at which NO gas is evolved and variation in the rate of evolution over time. Two primary shapes tested are a tablet and a sphere. The tablet theoretically provides the most uniform rate of dissolution while the sphere theoretically provides the higher total amount of NO gas evolution.

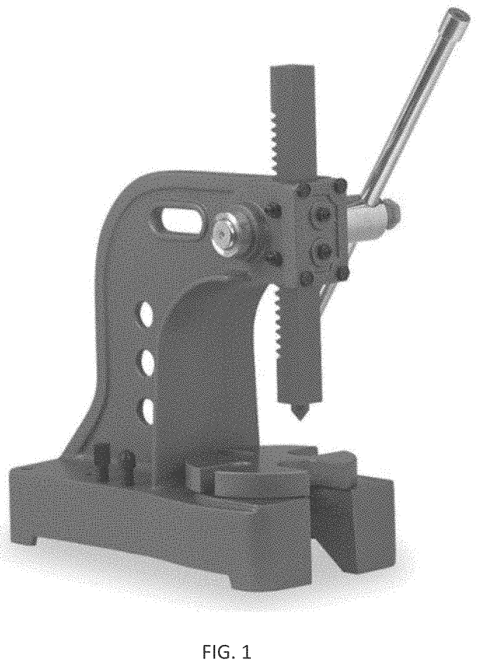

Molds could be created using metal 3D printing techniques or machining molds out of aluminum bar stock. An Arbor hand press like the one shown in FIG. 1 can be used to press tablets according to some embodiments.

An effervescent tablet will preferably have a rate of evolution of NO gas that can be determined. The variable factors in a designed tablet can include sodium nitrite concentration, tablet shape, and baking soda concentration in some embodiments.

The addition of other ingredients to the tablet to improve the treatment experience is also possible. The list of possible ingredients is large. Preferably ingredients do not have an interaction with the primary nitrite salt and acid. Possible additions can be Epsom salts to reduce swelling and essential oils for scent.

According to some embodiments, applications, methods and systems, a granular formulation of a nitrite salt, organic acid, and possibly other ingredients that dissolve into water may be used for a tablet or pellet that produces nitric oxide gas in a time release fashion. One purpose of this application may be to tailor the total amount and rate of NO gas evolution to specific treatment needs.

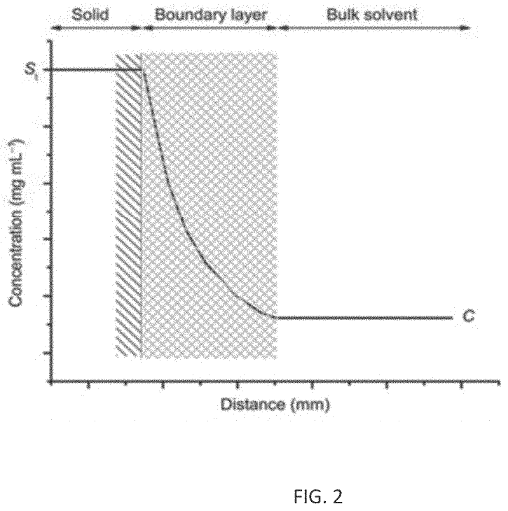

In some embodiments, a time release formulation can involve modifying the kinetics at which the sodium nitrite and organic acid dissolve into water. Therefore, a formulation may be used and/or engineered that slows down the diffusion of the soluble moieties from the solid surface through the boundary layer and into the bulk liquid. For example, see FIG. 2 showing a schematic of a boundary layer around a solid dissolving into a solvent.

According to some embodiments, there are three factors to consider in developing and utilizing a time release formulation. First, the sodium nitrite and organic acid may be pelletized to reduce the surface area to mass ratio of the salts. Second, the best material for coating the solutes can be determined. Third, the best method for coating the salt pellets can be determined and used. These factors can vary depending on the component material of the individual pellets in certain embodiments.

The sodium nitrite and citric acid can be dissolved in water before being mixed to create the NO gas. As such, the sodium nitrite and citric acid can be sourced as powders. Pelletized solutes may be obtained as source pelletized versions of the chemicals from a manufacturer or manufactures. Pelletized solutes may be developed in a suitable manner.

A suitable coating material can be utilized in certain embodiments. Requirements can include dissolving slowly, storing well, being safe as well as inexpensive, and that the coating material will preferably not interfere with the nitrite ion reacting with the acidic proton to produce NO gas.

Once the pellets of soluble salts are available, the pellets may be coated in any suitable manner to slow the production of NO gas. There is more than one way to coat pellets of material. According to some embodiments, the use of a tumble dryer in either a batch or continuous process can be used. For example, a coating process may utilize a benchtop or pilot scale tumble dryer. Factors to be considered in this effort can include time, temperature, and various machine settings.

According to some applications, the final step in a time release formulation can be to create several batches of coated soluble salt pellets. Each batch may have a different thickness of coated material. The NO gas evolution profile of each batch can be measured using an appropriate NO sensor. The results of the individual batch measurements can be used to calculate the amount of each batch that can be mixed to create a time release formulation that fits requirements in terms of evolution rate and duration.

According to some embodiments, the present disclosure relates to a system for using two separate mediums, or two separate solutions, to initiate the production of nitric oxide, especially in a water bath. Other baths are also possible. The production of nitric oxide forms bubbles that are filled with nitric oxide gas. In the water bath, the nitric oxide bubbles contact a user's skin enabling the nitric oxide to be absorbed by the user via transdermal absorption.

Once absorbed, the nitric oxide may be utilized by the user's body to treat a variety of ailments, including without limitation, diabetic ulcers, wounds, chronic wounds, circulatory issues, inflammation issues, and the like.

According to some embodiments, a first solution comprises a nitrite compound and a second solution comprises an acidic compound or a weak acid. The first and second solutions are added to a water bath, which initiates the production of nitric oxide in the water bath.

According to some embodiments, a first medium comprises a nitrite compound and a second medium comprises an acidic compound. The first and second mediums are placed in a water bath, which initiates the production of nitric oxide in the water bath. The first medium can be a tablet or pill that dissolves in the water bath. The second medium can be a tablet or pill that dissolves in the water bath. According to some embodiments, the first and/or second mediums are layered in a manner that controls the rates of production and release of nitric oxide in the water bath. According to some embodiments, the first and/or second mediums are compressed to a suitable hardness to allow for control of the rates of production and release of nitric oxide in the water bath.

According to some embodiments, a first medium comprises a nitrite compound or a nitrite solution, and a second medium comprises an acidic compound or an acidic solution. According to some embodiments, the first and second mediums are preferably configured as components of a single product, or single apparatus, which single product may be placed in a water bath. According to some embodiments, the single product is configured so as to dissolve and release the first and second mediums into the water bath.

According to some embodiments, a cylinder or tank containing nitric oxide gas is used to bubble nitric oxide into a water bath. According to some embodiments, the water bath comprises a suitable aqueous solution or aqueous medium, including without limitation, an aqueous solution that promotes and/or prolongs the availability of nitric oxide bubbles. According to some embodiments, a suitable bath container can be used. Preferably, in some embodiments, a bath container is configured and/or specifically adapted to enable the bubbling of nitric oxide into and through the water bath.

According to some embodiments, a water bath, or aqueous solution, preferably includes other components to help preserve the nitric oxide bubbles in the bath and/or to help promote the absorption of the nitric oxide gas by the user. For example, and not by way of limitation, the water bath in some applications preferably contains compounds that provide an environment suitable for preserving the nitric oxide bubbles (surfactant), for example, compounds to maintain or promote a certain pH (acids or bases, buffers of the same), compounds to maintain or promote a certain concentration of electrolytes (strongly dissolving salts, weakly dissolving salts), compounds to promote precipitation of undesirable components (compounds comprising a strongly soluble ion and a weakly dissolve ion such as sodium acetate, potassium carbonate, magnesium sulfate, or calcium phosphate), and/or the like.

According to some embodiments, an effervescent tablet comprises a nitrite salt, a weak acid, sodium bicarbonate, and another compound. According to some embodiments, an effervescent tablet comprises a suitable nitrite salt, a suitable weak acid, sodium bicarbonate, and other suitable compounds.

As used herein, “suitable” is a broad term and is used consistent with its plain and ordinary meaning to mean generally that which fits and has the qualities or qualifications to meet a given purpose, occasion, condition, function, or circumstance. Accordingly, in at least some instances relevant herein, the term “suitable” can convey one or more of the following traits, including but not limited to, safe, available, affordable, chemically appropriate, useful, disposable in a municipal sewer system, disposable in a septic system, FDA approved if possible, etc.

According to some preferred embodiments, systems, methods, and applications, the amount of water should preferably be enough to just cover the body part to be treated (for example, the foot or hand), the container to hold the water and body part preferably should be the minimum size to fit the body part to be treated and to allow water to exist between the body part and the container wall. Ideally, in some preferred embodiments, the amount of water is between about 1 and about 4 liters. According to some preferred embodiments, systems, methods, and applications, the range of the tablet size preferably is between about 100 mL to about 500 mL, and in some embodiments is preferably between about 10 mL to about 1 L.

The following examples of some embodiments are intended to be illustrative and not limiting.

According to some embodiments, sodium nitrite is used as a source of nitrite, which reacts with citric acid, or other acids, to produce nitric oxide. According to some embodiments, approximately one teaspoon of sodium nitrite is preferably used in an effervescent tablet embodiment. In some embodiments, one or more other nitrite salts can be used. For example, some embodiments comprise one or more nitrite salts, for example and not by way of limitation, sodium nitrite, potassium nitrite, calcium nitrite, lithium nitrite, magnesium nitrite, or the like. According to some embodiments, one or more nitrite salts from an alkali metal can be used. According to some embodiments, the amount of a nitrite salt component can be less than one teaspoon. According to some embodiments, the amount of a nitrite salt component can be greater than one teaspoon. According to some embodiments, the amount of nitrite salt components is between one-half teaspoon and two teaspoons. Other variations are also contemplated. For example, in some embodiments, the amount of nitrite salt components is between about one tenth of a teaspoon and about ten teaspoons, etc.

According to some embodiments, citric acid is used as a weak acid, which reacts with sodium nitrite, and/or other nitrite salts, to produce nitric oxide. According to some embodiments, approximately one-half cup of citric acid is preferably used in an effervescent tablet embodiment. According to some embodiments, one or more weak acids can be used. For example, some embodiments comprise one or more weak acids, for example and not by way of limitation, citric acid, ascorbic acid, salicylic acid, lactic acid, or the like. According to some embodiments, other acids can be used. According to some embodiments, acids can be used that require a certain amount of dilution before they are considered suitable for the intended purpose, for example and not by way of limitation, hypochlorous acid, or the like. According to some embodiments, the amount of a weak acid component can be less than one-half cup. According to some embodiments, the amount of a weak acid component can be greater than one-half cup. According to some embodiments, the amount of weak acid components is between one-quarter cup and two cups. Other variations are also contemplated. For example, in some embodiments, the amount of weak acid components is between about one tenth of a cup and about ten cups, etc.

According to some embodiments, sodium bicarbonate (baking soda) is used to react with the weak acid and provide effervescent action, bubbling, mixing, and helping to dissolve other components. According to some embodiments, approximately one cup of sodium bicarbonate is preferably used in an effervescent tablet embodiment. Other amounts are also suitable and contemplated. According to some embodiments, one or more other component chemicals can be used to react with acid to form carbon dioxide. For example and not by way of limitation, one or more carbonates can be used in some embodiments, one or more bicarbonates can be used in some embodiments, and/or one or more component chemicals that react to form a gas can be used in some embodiments, or the like. According to some embodiments, the amount of an effervescent component can be less than one cup. According to some embodiments, the amount of an effervescent component can be greater than one cup. According to some embodiments, the amount of one or more effervescent components is between one-half cup and two cups. Other variations are also contemplated. For example, in some embodiments, the amount of effervescent components is between about one tenth of a cup and about ten cups, etc.

According to some embodiments, Epsom salt (magnesium sulfate heptahydrate) is preferably used to increase the osmolality of the water bath and can reduce swelling in the body part being immersed. According to some embodiments, approximately one-half cup of Epsom salt is preferably used in an effervescent tablet embodiment. Other amounts are also suitable and contemplated. According to some embodiments, one or more other components that dissolve completely in water can be used. For example and not by way of limitation, one or more alkali metal salts of chlorine, sulfate, nitrate, iodine, bromine, and/or sodium borate (borax) can be used in some embodiments, or the like. According to some embodiments, the amount of a component can be less than one-half cup. According to some embodiments, the amount of a component can be greater than one-half cup. According to some embodiments, the amount of one or more components is between one-quarter cup and two cups. Other variations are also contemplated. For example, in some embodiments, the amount of a component is between about one tenth of a cup and about ten cups, etc.

According to some embodiments, borax and/or other chemicals are preferably used to soften the water to reduce precipitates and/or increase the speed of nitric oxide production. According to some embodiments, approximately one-half cup of borax is preferably used in an effervescent tablet embodiment. Other amounts are also suitable and contemplated. According to some embodiments, the amount of a softener component can be less than one-half cup. According to some embodiments, the amount of a softener component can be greater than one-half cup. According to some embodiments, the amount of one or more softener components is between one-quarter cup and two cups. Other variations are also contemplated. For example, in some embodiments, the amount of a softener component is between about one tenth of a cup and about ten cups, etc.

According to some embodiments, corn starch is preferably used as a packaging agent or filling agent which is compressible and helps to form the tablet. According to some embodiments, approximately one-half cup of corn starch is preferably used in an effervescent tablet embodiment. Other amounts are also suitable and contemplated. According to some embodiments, one or more anti-conglomerate agent, and/or packaging agent, and/or ultra-fine powder can be used. For example, and not by way of limitation, ultra-fine silica can be used in some embodiments, and/or bentonite can be used in some embodiments, or the like. According to some embodiments, the amount of one or more packing agent and/or filling agent components can be less than one-half cup. According to some embodiments, the amount of one or more packing agent and/or filling agent components can be greater than one-half cup. According to some embodiments, the amount of one or more packing agent and/or filling agent components is between one-quarter cup and two cups. Other variations are also contemplated. For example, in some embodiments, the amount of one or more packing agent and/or filling agent components is between about one tenth of a cup and about ten cups, etc.

According to some embodiments, coconut oil, and/or another oil, may be used as a binder, or binding agent, and may also help moisturize the skin. According to some embodiments, approximately two tablespoons of coconut oil is preferably used in an effervescent tablet embodiment. Other amounts are also suitable and contemplated. According to some embodiments, one or more oils or compounds may be used as a binder and/or moisturizer. For example, and not by way of limitation, one or more of a coconut oil, olive oil, mineral oil, essential oils, or the like can be used in some embodiments. According to some embodiments, a suitable compound that liquefies under the pressure of making the tablet can be used. For example, in some embodiments, compounds with molecules that have high intermolecular forces like PTFE (polytetrafluoroethylene), PVDF (polyvinylidene fluoride), PIB (polyisobutylene) can liquefy under pressure in some applications.

According to some embodiments, an oil preferably provides one or more of the following advantageous features: fragrance, moisturizer, and/or binder, or another advantageous purpose. According to some embodiments, the amount of one or more oils, binders, moisturizer, and/or fragrance components can be less than two tablespoons. According to some embodiments, the amount of one or more oils, binders, moisturizer, and/or fragrance components can be more than two tablespoons. According to some embodiments, the amount of one or more oils, binders, moisturizer, and/or fragrance components is between one tablespoon and four tablespoons. Other variations are also contemplated. For example, in some embodiments, the amount of one or more oils, binders, moisturizer, and/or fragrance components is between about one tenth of a tablespoon and about ten tablespoons, etc.

According to some embodiments, a desiccant is preferably used to increase the shelf life of a tablet. For example and not by way of limitation, according to some embodiments, one or more of the following components form at least a portion of the embodiment: silica gel, calcium sulfate, calcium chloride, zeolites, bentonite clay, or the like. According to some embodiments, a colorant like cobalt chloride, for example, could be added to the desiccant to indicate when the desiccant is saturated.

According to some embodiments, other additives are preferably used with an effervescent tablet. Compounds that provide a fragrance are advantageously used in some embodiments. According to some embodiments, a fragrance component preferably does not interfere or detract from allowing for and/or promoting transdermal absorption of nitric oxide.

According to some embodiments, it is contemplated that certain components of an effervescent tablet can be substituted for other components and or provided in combination with other components. For example, corn starch can be replaced and/or supplemented with a finely ground powder of a cationic surfactant, such as, for example, Cocamidopropyl Betaine, behentrimonium metholsulfate, or the like. According to some embodiments, the powder preferably is finely ground to improve binding of the tablet. Such components advantageously also promote the formation of bubbles and/or a foam on top of the water bath so that nitric oxide produced in the water bath may go into the bubbles or foam, as opposed to simply escaping into the surrounding atmosphere. According to some embodiments, bubbles forming a layer at a surface of the water bath preferably form a barrier or bubble cover to hinder nitric oxide gas from escaping to the atmosphere. Other suitable layers or covers are also contemplated. For example, covers at the surface or layers suspended within the water bath can act to contain nitric oxide for an extended period and increase the amount of nitric oxide that can be delivered to the user of the bath.

According to some embodiments, one or more components is preferably coated with a water-soluble compound to control the rate of release, or the rate of availability of the coated component. In some embodiments, the thickness of the coating is selected and designed to preferably control the rate of reaction, or when the reaction begins. According to some embodiments, one or more coatings can be used. According to some embodiments, for example and not by way of limitation, polyvinyl alcohol can be used, or the like.

According to some embodiments, when making an effervescent tablet, it can be advantageous to provide that some or all powdered compounds are sufficiently comminuted to facilitate the production of a better-quality tablet. According to some embodiments, for example, powders that can pass through approximately 100-350 mesh are preferred. According to some embodiments, the pressure used to form the effervescent tablet preferably is selected and designed to controls and/or affect the release rate and the rate of nitric oxide production in a preferred manner. According to some embodiments, the pressure applied should be minimally sufficient to create a tablet. According to some embodiments, the pressure applied can be increased and/or adjusted for certain advantageous purposes. For example, according to some embodiments, more pressure is preferably applied to slow the reaction rate for a given tablet. According to some embodiments, one or more hydraulic presses is provided and/or used for pressing a tablet or plurality of tablets as described herein. Also, according to some embodiments, systems, and methods, a mold for forming one or more tablets is preferably used. According to some embodiments, the mold is specially designed and configured to advantageously be adapted to withstand the physical characteristics of the tablet, the acidic nature of the tablet, and the use of acids such as citric acid. According to some embodiments, manufacturing equipment for forming tablets are preferably formed from advantageous materials that are durable and long lasting to withstand the physical characteristics of the tablet, the acidic nature of the tablet, and the use of acids. According to some embodiments, manufacturing equipment for forming tablets are preferably formed from advantageous materials that are durable and long lasting to withstand the physical characteristics of the tablet, the acidic nature of the tablet, and the use of acids such as, for example, stainless steel (302 and 316), Durimet™, Hastelloy™, Titanium, etc.

According to some embodiments, a tablet may have multiple layers. According to some embodiments, individual layers are advantageously fabricated and/or formulated to provide certain amounts of nitric oxide over varying durations and/or at various stages of the bath. For example, according to some embodiments, a tablet is preferably fabricated to include delays between nitric oxide production. According to some embodiments, a tablet is preferably fabricated to include variable amounts of nitric oxide production. According to some embodiments, a tablet is preferably fabricated to provide particular concentrations of nitric oxide production. According to some embodiments, a tablet is preferably fabricated to provide nitric oxide therapy over a span of about one to about thirty minutes. According to some embodiments, a tablet is preferably fabricated to provide nitric oxide therapy over a span of less than about one minute. According to some embodiments, a tablet is preferably fabricated to provide nitric oxide therapy over a span of more than about thirty minutes. According to some embodiments, a tablet is preferably fabricated to provide nitric oxide therapy over a span of about up to about forty minutes. According to some embodiments, a tablet is preferably fabricated to provide nitric oxide therapy over a span of up to about forty-five minutes. According to some embodiments, a tablet is preferably fabricated to provide nitric oxide therapy over a span of up to about fifty minutes. According to some embodiments, a tablet is preferably fabricated to provide nitric oxide therapy over a span of up to about one hour. According to some embodiments, a tablet is preferably fabricated to provide nitric oxide therapy over a span of up to about two hours. Advantageously, the use of select layers and/or coatings may enable a wide variety of options for a tablet.

According to some embodiments, the components of a tablet preferably comprise an increased amount of surfactant, thus making the tablet like a bar of soap. According to some specific compositions or formulations of the bar embodiments, and/or slab embodiments, are selected according to desired characteristics, with the result that some bar embodiments preferably provide another form of topical nitric oxide therapy. For example, according to some embodiments, as a bar is being used, the bar produces nitric oxide that is transdermally absorbed by the user. According to some embodiments, the pressure required to form a bar and or slab can be adjusted to affect the release rate and the rate of nitric oxide production.

According to some embodiments, a pod or a pouch is preferably utilized to provide the reagents and/or compounds needed to initiate nitric oxide therapy in a bath. For example, and not by way of limitation, according to some embodiments, a pod or a pouch is preferably comprised of polyvinyl alcohol. In some embodiments, another suitable compound can be used. According to some embodiments, the pouch is preferably filled with reagents necessary to produce nitric oxide therapy in a water bath. According to some embodiments, a pouch preferably includes one or more pockets, or sub-pouches. Various configurations of pouches, pockets, pods, and sub-compartments can be used depending on a desired configuration, orientation, and or treatment plan. In some embodiments, a pouch or pocket may be formed and sealed in any suitable manner. According to some embodiments, contents of a pod, pouch, pocket, and/or sub-compartment can be in a liquid medium, in a powdered medium, in a solid medium, and/or in a gaseous medium. According to some embodiments, powdered reagents and compounds used in a pouch or pocket do not need to be milled or ground, or provided in a finer particle size, as compared to reagents or compounds used to make tablets. According to some embodiments, powdered reagents and compounds used in a pouch or pocket are preferably milled or ground, or provided in a fine particle size for any number of reasons, including without limitation, ease of manufacturing and consistency in treatment and/or production.

According to some embodiments, a pod or pouch does not include fillers, like corn starch and/or oil. According to some embodiments, fillers like corn starch and/or oil may not be included because the pod or pouch reagent components do not need to be in the form of a pill or tablet. However, according to some embodiments, fillers like corn starch and/or oil are preferably included in view of a desired configuration and/or other physical properties. According to some embodiments, a pouch preferably includes the reagents in a powdered form for quicker production of nitric oxide. According to some embodiments, a pouch preferably includes the reagents in a tablet form for slower, or more controlled, production of nitric oxide.

According to some embodiments, a nitric oxide therapy bath is preferably configured to form a film or coating on the surface of the bath. According to some embodiments, for example, and not by way of limitation, a surfactant film preferably forms on the surface of a water bath. According to some embodiments, such a film acts to limit nitric oxide produced in the water bath from simply escaping to the surrounding environment by forming a surface barrier. According to some embodiments, nitric oxide gas is advantageously conserved to an extent by the cover created. According to some embodiments, at least a portion of the nitric oxide gas in the surfactant film is absorbed transdermally by the user. According to some embodiments, a majority of the nitric oxide gas in the surfactant film is absorbed transdermally by the user.

According to some embodiments, a bath is preferably equipped with a cage, and/or a vented tray. According to some embodiments, a cage and/or a vented tray can be used with a water bath in a manner that enables a tablet, or a pouch, to be effectively trapped in place at the bottom of the water bath. According to some embodiments, applications, systems, and methods, when one or more tablets, pods, and/or pouches, are placed at the bottom of a water bath, and effectively trapped in position, a user may then position their foot, leg, hand, arm, and/or other body part or body surface, more directly over the source of the nitric oxide gas. Advantageously, according to some embodiments, a user can position a body part or surface over the cage and/or vented tray comprising one or more tablets, pods, and/or pouches while effervescing and producing nitric oxide gas. According to some embodiments, systems and methods, the orientation of the user within the bath relative to the active location of production of nitric oxide gas advantageously increases the transdermal absorption of nitric oxide gas by the user.

According to some embodiments, a tablet or pouch is preferably configured to provide a significant amount of foam. According to some embodiments, a tablet or pouch is preferably configured to provide a significant amount of foam and is specifically adapted and designed for use in a relatively smaller amount of liquid. According to some embodiments, a significant amount of foam is produced in the bath, wherein the foam comprises bubbles containing nitric oxide gas. According to some embodiments, a user's foot, or other body part or body surface, is preferably essentially encased in a “foam bath.” For example, according to some embodiments, a water bath preferably is comprised of approximately two cups of water and one or more tablets and/or pouches specifically formulated to produce a significant amount of bubbles containing nitric oxide gas, or a nitric oxide foam. Similarly, according to some embodiments, systems, and methods, a bath container or apparatus preferably is configured, arranged, designed and/or adapted to provide additional agitation of the water to facilitate the production of a significant amount of foam. For example, mechanisms for vibration, mechanical mixing, shaking, rotating, jets, and/or other mechanisms can be provided in some systems, embodiments, and methods to increase foam production.

Additional aspects of the present disclosure may be embodied in other specific forms without departing from its fundamental functions or essential characteristics of this disclosure. The described embodiments are to be considered in all respects only as illustrative, and not restrictive. All changes which come within the meaning and range of equivalency of the illustrative embodiments are to be embraced within their scope.

According to some embodiments, systems, methods, and applications, a method for producing nitric oxide in a bath comprises providing water in a bath container. The method can include providing a nitrite medium. The method can include providing an acidic medium. The method can include adding the nitrite medium and the acidic medium to the water thereby initiating the production of nitric oxide bubbles in the water within the bath container. The method can include placing a portion of a user's body in the water within the bath container. The method can include use of a vented tray.

According to some embodiments, systems, methods, and applications, a system is provided for producing nitric oxide bubbles in an aqueous solution for treating affected areas of skin and wounds, wherein the nitric oxide bubbles deliver nitric oxide gas to the affected area and promote the healing response of the body.

According to some embodiments, systems, methods, and applications, effervescent tablets, pills, pods, and/or pouches can be manufactured, provided, and used for bath systems and methods.

According to certain embodiments and applications, advances in the use of a therapeutic bath have been developed. According to some embodiments, tablets, pills, pods, pouches, and other containers have been developed that are specially adapted for the production and delivery of nitric oxide gas therapy in a bath system. Various features, advantages and embodiments are discussed herein, as examples, without limitation, and also described and disclosed in the materials incorporated by reference herein.

According to some embodiments, a tablet of a nitrite salt, an organic acid, and other ingredients can be added to a bath for the care of wounds, neuropathy, and ulcers. The bath can be a foot bath, a hand bath, a bath for any body part of a user. Some embodiments are directed to several advantageous versions of a tablet having many features and desired attributes. Accordingly, certain features and variations will be described herein, but they are examples and non-limiting.

According to some embodiments, the primary action of creating NO gas is particularly advantageous. The tablet preferably contains sodium nitrite to create the nitrite ion when the salt dissolves in water. The tablet preferably also contains citric acid and/or ascorbic acid to provide the acidic proton to react with the nitrite ion to create NO gas. Other materials are also contemplated as discussed herein. According to some advantageous embodiments, ascorbic acid is used and thereby the coproduction of NO2 gas is minimized.

According to some embodiments, the creation of NO gas makes the tablet dissolve in an effervescent manner. According to some embodiments, the reactants preferably react to create relatively large amounts of gas bubbles. According to some embodiments, applications, methods, and treatments, providing relatively large amounts of gas bubbles preferably and advantageously can provide for one or more of: better mixing of the ingredients in the bath, a better treatment experience for the user, and/or a more pleasing experience overall. According to some other embodiments, application, methods, and treatments, providing relatively smaller amounts of gas bubbles is preferred for some treatments and compositions.

According to some embodiments, an advantageous system provides for a more vigorous effervescent action by providing baking soda (sodium bicarbonate) to the tablet ingredients. In some embodiments, an amount of baking soda in the composition can have a corresponding and/or commensurate amount of a weak acid, such as, for example, citric acid and/or ascorbic acid, whereby the ingredients can act to offset the baking soda neutralization.

According to some embodiments, some features of the tablet are configured and designed so as to provide the required robustness of the tablet to shipping and other handling. According to some embodiments, the tablet preferably is formulated with a binding agent specified for the pressure and time spent under pressure in the press. In some embodiments, the tablet preferably is packaged in shrink wrap, or another suitable cover, preferably in such a way as to protect it.

The shape of the tablet can affect the effervescent tablet application in two ways. According to some embodiments, the shape of the table is adapted and configured to control the rate at which the tablet dissolves in water. Therefore, the shape acts to control the rate at which NO gas is evolved and variation in the rate of evolution over time. Many different shapes are contemplated. According to some embodiments, one primary shape is in the form of a tablet. According to some embodiments, one primary shape is in the form of a sphere. According to some embodiments, one primary shape is in the form of a bar. According to some embodiments, one primary shape is in the form of a ring. According to some embodiments, one primary shape is in the form of a cube. According to some embodiments, one primary shape is in the form of a pellet. According to some advantageous embodiments, the tablet shape preferably provides a relatively uniform rate of dissolution. According to some advantageous embodiments, the sphere shape preferably provides a relatively higher total amount of NO gas evolution in some configurations.

According to some embodiments, molds are preferably created using metal 3D printing techniques and/or machining out of aluminum bar stock. According to some embodiments and applications, any suitable press can be used. In some embodiments, a small-scale press can be used. In other embodiments, a large-scale press can be used. One example of a suitable small-scale press is an Arbor hand press as discussed herein with reference to FIG. 1.

According to some embodiments, test procedures for evaluating the effectiveness of effervescent tablets have been developed. According to some applications, factors and variables are monitored, such as, for example, the rate of evolution of NO gas, the sodium nitrite concentration, the tablet shape, and/or the baking soda concentration. According to some applications, multiple formulations and variations are tested.

According to some embodiments, other ingredients are preferably added to the tablet to improve the treatment experience. According to some embodiments, the added ingredients preferably do not have an adverse interaction with the primary nitrite salt and acid. According to some embodiments, added components can include one or more of Epsom salts to reduce swelling and/or oils, including essential oils for scent, texture, and/or binding.

According to some embodiments, a pod and/or pouch configuration can be used. For example, as opposed to the pressed tablet configuration, some embodiments and systems comprise for example reactive components enclosed in a water-soluble membrane and separated therein by a dissolvable inner wall such that when immersed in water both the outer packet wall and the inner wall dissolve allowing the reactive components to mix and react to produce an effervescent result. According to some embodiments, one or more pods can comprise at least two reactants. According to some embodiments, one or more pods can comprise a single reactant. According to some embodiments, one or more pods can be added to the bath. In some embodiments selection of the number and type of pods can allow for various desired treatment profiles.

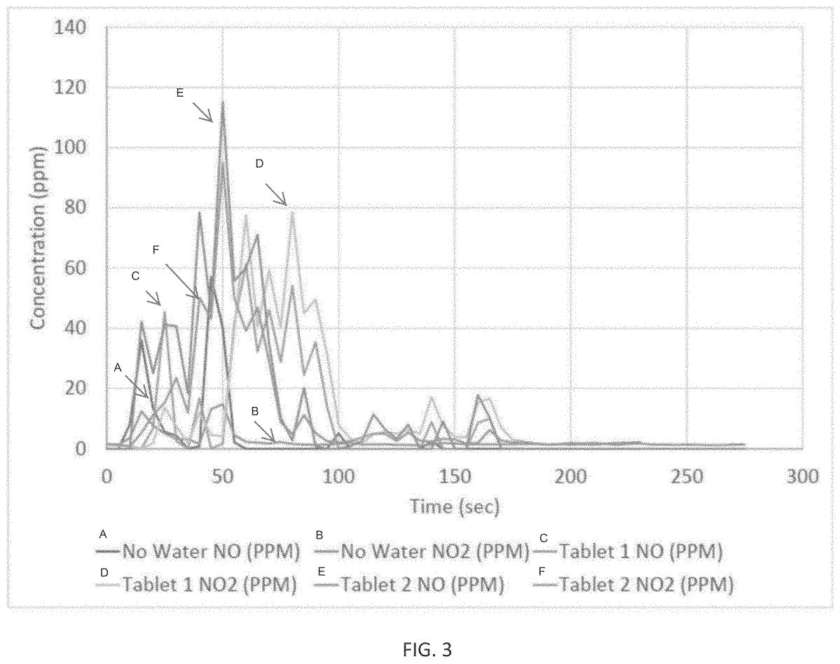

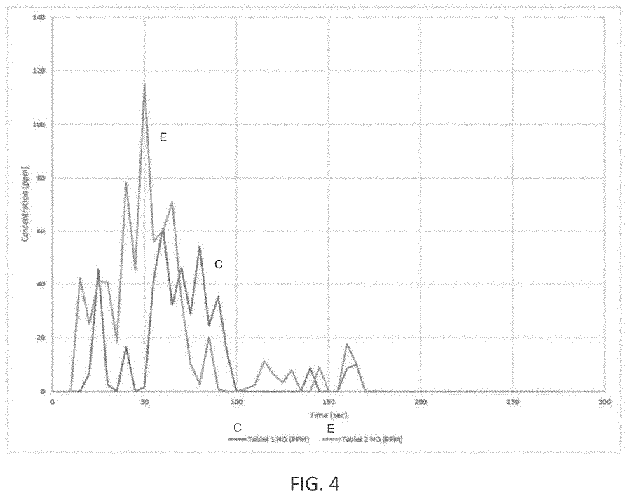

According to some embodiments, systems, methods, aspects, and applications, testing regarding the concentration in parts per million of NO and/or NO2 gas generated over time by the use of first and second tablets as described herein can provide data to reflect the distribution of gas over time, including with and without water in some applications. For example, FIG. 3 shows relative concentrations over time for NO and NO2 with respect to no water embodiments, and embodiments with first and second tablets respectively. FIG. 4 shows a comparison of the concentrations of NO over time for a first tablet and a second tablet as described herein. Testing data and charts supporting FIGS. 3 and 4 have been incorporated herein by reference.

A user can obtain the benefits of nitric oxide therapy by utilizing a tablet of reactants designed to deliver nitric oxide gas to the surface of the user's skin. In some embodiments, a foot bath may include a tub that may hold a suitable amount of water that may allow for a user's feet to be immersed in the water. At least one tablet of reactants may be placed or incorporated with the water. In some embodiments, each tablet placed in the water may be composed of different reactants. The addition of the tablets to the water may initiate a reaction that may produce nitric oxide gas. The tablets of reactants may be effervescent, which may result from the creation of nitric oxide gas. In some embodiments, sodium bicarbonate, or baking soda, may be utilized to help promote or cause effervescence. A more vigorous effervescent effect may be achieved by the addition of baking soda or sodium bicarbonate to the tablet components. The tablets of reactants may include other components, such as Epsom salts (e.g., to reduce swelling), fragrances (e.g., for scent), etc. In some embodiments, a commensurate amount of citric acid or ascorbic acid can be added to the tablet of reactants ingredients which may offset the baking soda neutralization.

According to some embodiments, the tablets of reactants may include a nitrite compound or medium and an acidified, or acidic, compound, or medium. The nitrite compound or medium can be formulated and/or formed in any suitable manner and the concentration of reactants can be adjusted to obtain sufficient concentration of nitric oxide through the intended reaction.

In certain embodiments, a first compound, or nitrite compound, may include sodium or potassium nitrite. This can create a nitrite ion upon the salt dissolving in water. In certain embodiments, a second compound, or acidified compound, may include ascorbic acid or citric acid. The acidic proton can react with the nitrite ion and may create nitric oxide gas. The use of ascorbic acid as the acidified compound or medium may produce less or minimal amounts of nitrogen dioxide, or even negligible amounts of nitric dioxide. This may promote a purer production of nitric oxide gas.

According to some embodiments, a layer of foam may be formed on the top of the foot bath. Nitric oxide gas may have an affinity to capillary beds, which may result in the nitric oxide gas virtually seeking out, or being attracted to, capillaries and capillary beds to be absorbed into them. The layer of foam may help to trap nitric oxide gas that can escape the water of the foot bath before it is absorbed by a user.

According to some embodiments, a bath or soak may be engineered to allow for a bottom compartment to be structurally separated from a space where a user's feet may be. The bottom compartment, or lower compartment, may be where the reactants are used to initiate the production of nitric oxide gas and a nitric oxide foam. The nitric oxide foam may include bubbles that contain the nitric oxide gas. The number of bubbles produced, or present may affect the mixability of the bath or soak components and general treatment experience. The nitric oxide foam may fill or share the space where the user's feet would be. This may be considered a foam bath or soak.

According to some embodiments, the shape of the tablets of reactants may be formed in a manner to obtain a sufficient surface area to volume ratio. For example, a tablet of reactants may be formed in various shapes, such as a disc, sphere, cube, cylinder, etc. A tablet of reactants of any shape may be comprised of various reactants, such as sodium bicarbonate, fragrance materials, etc. The shape of the tablets of reactants may affect the effervescent effect. For example, the shape of the tablet of reactants may control the rate at which the tablets of reactants dissolves in water, which may affect the rate at which nitric oxide gas is evolved and the variation in the rate of evolution over time. In some embodiments, the shape of the tablet of reactants may allow for the tablet to dissolve in a uniform rate or in a manner which provides for a higher total amount of nitric oxide gas evolution, for example, when the tablet of reactants may be in a spherical shape. In some embodiments, the robustness of the tablet of reactants may be determined by what may be required for the shipping and handling of the tablet of reactants. The tablet of reactants may be formulated with a binding agent, which may be specified for the pressure and time spent under pressure in a press. The tablet of reactants may be packaged in a way as to protect the tablet of reactants with a layer of protection, such as shrink wrap. A mold may be used to create the shape of the tablets of reactants. The mold may be created using a metal 3D printing technique or machining molds from aluminum bar stock. For example, a press may be used to create the shape of the tablets of reactants as disclosed herein.

According to some embodiments, the tablets of reactants may be kinetically modified to slow down or speed up the diffusion of soluble moieties from the solid surface of the tablets of reactants through the boundary layer and into the bulk liquid, as shown in FIG. 2, which may affect the rate at which the tablets of reactants dissolves in water. In some embodiments, the tablets of reactants may be granular, pelletized, and coated in a manner that may allow for the granules to include a time-release function. For example, the tablets of reactants may be coated with polyethylene glycol, or any suitable coating. The coating may substantially dissolve before the reactants come in contact with a solution that may activate the respective reactants. A suitable coating material may not include an ingredient that may react with the reactants being coated. In certain embodiments, one coating material may be suitable for nitrite reactants. In certain embodiments, a separate coating material may be suitable for acidified reactants. The thickness of the coating on the reactants may vary, which may impact the time it takes to dissolve the coating. The coated tablets of reactants with varying thicknesses may be utilized to prolong and/or maintain nitric oxide gas production. The coated tablets of reactants with varying thicknesses may be portioned so that more nitric oxide gas may be produced at time interval as compared to other time intervals. The tablets of reactants may be coated by using a tumble dryer in either a bath or continuous process.

Testing was conducted to evaluate various aspects of using a pod of powder encased in polyvinyl alcohol (PVA). According to some embodiments, a pod may contain various compounds associated with the production of nitric oxide when the pod is placed in water. Testing was conducted to measure the nitric oxide release profile of the gas. Testing was also conducted to evaluate the effects of adding sodium bicarbonate to a pod, or reaction mixture, including determining whether more gas is produced, i.e., carbon dioxide. Also, the effects of adding a surfactant were evaluated, including determining whether a slurry of bubbles, or a foam, or a lather could capture the carbon dioxide (CO₂) and nitric oxide.

According to a testing method, a powder mixture of reactants or compounds is placed on one-half of a Medline™ towel. Water is sprayed on the towel while minimizing the amount of water that comes into contact with the powder. A sheet of 7 mm thick polyvinyl alcohol is placed over the powder and wet towel. The polyvinyl alcohol is then pressed down around the powder to seal the polyvinyl alcohol to the towel, encasing the powder. This creates a suitable pod. Table 1 shows an example of a powder mixture.

TABLE 1

| Chemical | |
|---|---|
| (powders - not milled) | Mass (g) |
| Citric Acid | 8.0 |
| Sodium Bicarbonate | 9.0 |
| Sodium Lauryl Sulfoacetate | 1.5 |
| Sodium Nitrite | 3.0 |

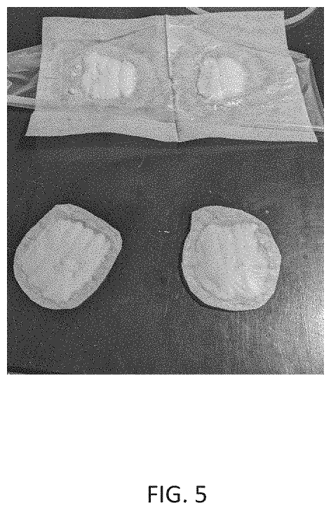

The pod is allowed to dry overnight. The excess towel and polyvinyl alcohol is trimmed from the pod the next day. FIG. 5 shows examples of untrimmed and trimmed pods made as described herein. Thus, the polyvinyl alcohol and the Medline™ towel create an envelope around the reactants that are contained within the pod. The pod can contain, or envelop, any and all suitable reactants.

The pod is placed in 1 L (one liter) of room temperature well water inside a large plastic sampling chamber. The NOx concentration is measured for 25 minutes. At the end of the testing, the water is discarded and the sampling chamber (a glass bowl) is cleaned. The next sample is taken or tested when the concentration of NOx is below 5 ppm.

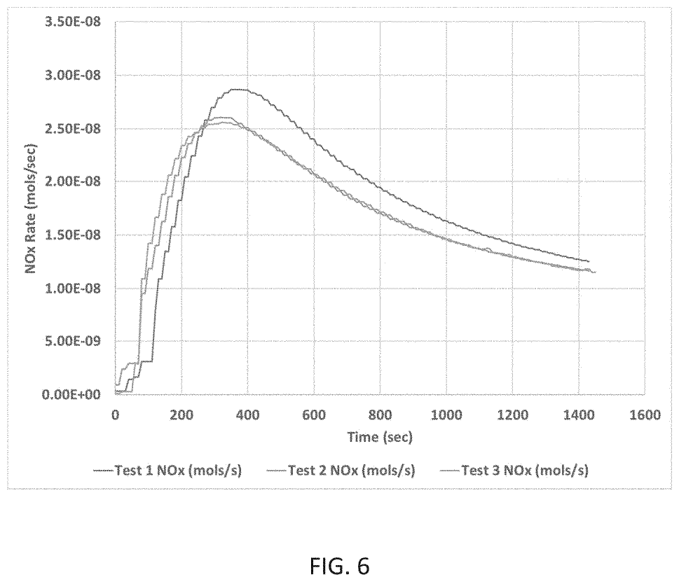

The release of nitric oxide was measured for three separate pods. The traces of the tests are shown in FIG. 6. The average temperature, total NOx, and maximum NOx measured are reproduced in Table 2.

TABLE 2

| | Average Temperature | Total NOx | Maximum NOx |
|---|---|---|---|
| Test | (° C.) | (mols) | (mols/sec) |
| 1 | 27.2 | 2.58E−05 | 2.87E−08 |
| 2 | 28.5 | 2.42E−05 | 2.61E−08 |
| 3 | 29.0 | 2.46E−05 | 2.56E−08 |

The pod as described, produced, and tested is a suitable device to affect a nitric oxide bath, or footbath. A pod may have different shapes, which shapes may affect the rate of nitric oxide release. For example, a pod may be shaped like a disc, a tablet, a sphere, a cube, a cylinder, or the like.

Testing was conducted to evaluate and measure the production of NOx in mixed acidified nitrite foam for solutions of varying sodium citrate buffer concentrations (differing pH levels). The higher the pH, the lower the total and maximum NOx measured over five minutes. The nitric oxide release profile of effervescent tablet formulations, with certain variations, was measured.

According to a testing method, an effervescent tablet was pressed into a large tablet. An effervescent table may have a general formulation comprising 195 grams of sodium bicarbonate, 123 grams of citric acid, 120 grams of Epsom salts, 54.8 grams of olive oil, 72.2 grams of corn starch, and 83.4 grams of sodium nitrite. In some embodiments, cocamidopropyl betaine (CAPB) may be added to the formulation. The release profile of four formulations were measured and are shown in Table 3.

TABLE 3

| | DI Water | Tablet | CAPB | Total NO | Max NO |
|---|---|---|---|---|---|
| Test # | (g) | (g) | (g) | (mols in 5 min) | (mols/sec) |
| 1 | 200 | 1.14 | 0 | 4.99E−07 | 1.84284E−09 |
| 2 | 200 | 2.9 | 0 | 1.80004E−06 | 6.86E−09 |
| 3 | 200 | 3.06 | 0.03 | 1.9163E−06 | 7.05857E−09 |
| 4 | 200 | 3.05 | 1 | 4.9209E−07 | 1.82796E−09 |

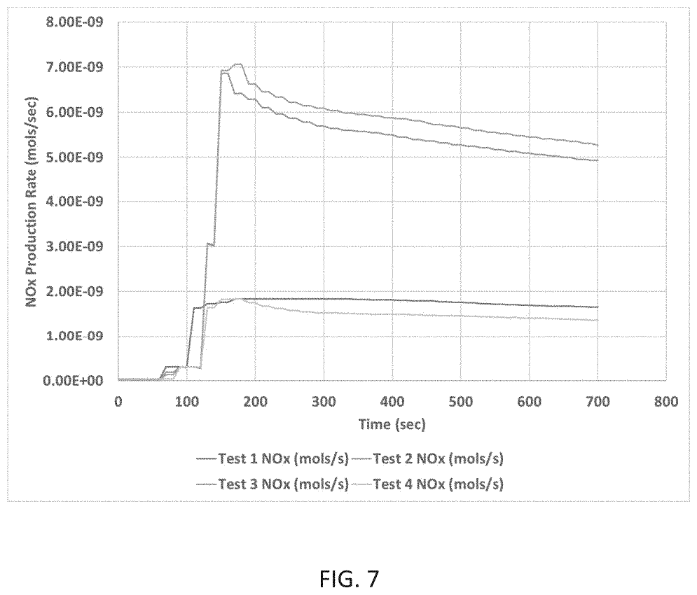

FIG. 7 is a graph illustrating the results of Table 3.

Accordingly, at least to the first order, the amount of nitric oxide released is directly proportional to the mass of the tablet added to the water. For example, see the results of Test 1 and Test 2, as shown in Table 3. The amount of nitric oxide measured by the analyzer is inversely proportional to the concentration of surfactant added to the water before the tablet. For example, see the results of Test 1, Test 3, and Test 4 in Table 3. At some minimum concentration of surfactant between 0.03 g/200 mL of water and 1.0 g/200 mL of water, the evolved carbon dioxide gas creates a slurry of bubbles, or a foam, that traps the nitric oxide gas as it leaves the water.

While the above detailed description has shown, described, and pointed out novel features of the development as applied to various embodiments, it will be understood that various omissions, substitutions, and changes in the form and details of the devices illustrated can be made by those skilled in the art without departing from the spirit of the development. As will be recognized, the present development can be embodied within a form that does not provide all the features and benefits set forth herein, as some features can be used or practiced separately from others. All changes which come within the meaning and range of equivalency of the claims are to be embraced within their scope. Additional disclosure and relevant information supporting this application is also provided in the following patents and applications, each of which is hereby incorporated by reference herein in its entirety: U.S. Pat. Nos. 7,939,045; 9,138,707; 10,517,817; 8,685,467; 10,052,348; 8,720,436; 8,434,475; 9,649,467; 8,501,090; and U.S. patent application Ser. Nos. 17/727,651, 17/727,630, 17/494,180, are each incorporated by reference herein and made part of this disclosure.

The foregoing description details some embodiments of the systems, devices, and methods disclosed herein. It will be appreciated, however, that no matter how detailed the foregoing appears in text, the systems, devices, and methods can be practiced in many ways. As is also stated above, it should be noted that the use of particular terminology when describing certain features or aspects of the invention should not be taken to imply that the terminology is being re-defined herein to be restricted to including any specific characteristics of the features or aspects of the technology with which that terminology is associated.

It will be appreciated by those skilled in the art that various modifications and changes can be made without departing from the scope of the described technology. Such modifications and changes are intended to fall within the scope of the embodiments. It will also be appreciated by those of skill in the art that parts included in one embodiment are interchangeable with other embodiments; one or more parts from a depicted embodiment can be included with other depicted embodiments in any combination. For example, any of the various components described herein and/or depicted in the Figures can be combined, interchanged, or excluded from other embodiments.

With respect to the use of substantially any plural and/or singular terms herein, those having skill in the art can translate from the plural to the singular and/or from the singular to the plural as is appropriate to the context and/or application. The various singular/plural permutations can be expressly set forth herein for sake of clarity.

It will be understood by those within the art that, in general, terms used herein are generally intended as “open” terms (e.g., the term “including” should be interpreted as “including but not limited to,” the term “having” should be interpreted as “having at least,” the term “includes” should be interpreted as “includes but is not limited to,” etc.). It will be further understood by those within the art that if a specific number of an introduced claim recitation is intended, such an intent will be explicitly recited in the claim, and in the absence of such recitation no such intent is present. For example, as an aid to understanding, the following appended claims may contain usage of the introductory phrases “at least one” and “one or more” to introduce claim recitations. However, the use of such phrases should not be construed to imply that the introduction of a claim recitation by the indefinite articles “a” or “an” limits any particular claim containing such introduced claim recitation to embodiments containing only one such recitation, even when the same claim includes the introductory phrases “one or more” or “at least one” and indefinite articles such as “a” or “an” (e.g., “a” and/or “an” should typically be interpreted to mean “at least one” or “one or more”); the same holds true for the use of definite articles used to introduce claim recitations. In addition, even if a specific number of an introduced claim recitation is explicitly recited, those skilled in the art will recognize that such recitation should typically be interpreted to mean at least the recited number (e.g., the bare recitation of “two recitations,” without other modifiers, typically means at least two recitations, or two or more recitations). Furthermore, in those instances where a convention analogous to “at least one of A, B, and C, etc.” is used, in general such a construction is intended in the sense one having skill in the art would understand the convention (e.g., “a system having at least one of A, B, and C” would include but not be limited to systems that have A alone, B alone, C alone, A and B together, A and C together, B and C together, and/or A, B, and C together, etc.). In those instances where a convention analogous to “at least one of A, B, or C, etc.” is used, in general such a construction is intended in the sense one having skill in the art would understand the convention (e.g., “a system having at least one of A, B, or C” would include but not be limited to systems that have A alone, B alone, C alone, A and B together, A and C together, B and C together, and/or A, B, and C together, etc.). It will be further understood by those within the art that virtually any disjunctive word and/or phrase presenting two or more alternative terms, whether in the description, claims, or drawings, should be understood to contemplate the possibilities of including one of the terms, either of the terms, or both terms. For example, the phrase “A or B” will be understood to include the possibilities of “A” or “B” or “A and B.”

The term “comprising” as used herein is synonymous with “including,” “containing,” or “characterized by,” and is inclusive or open-ended and does not exclude additional, unrecited elements or method steps.

The above description discloses several methods of manufacture and materials of the present development. This development is susceptible to modifications in the methods and materials, as well as alterations in the fabrication methods and equipment. Such modifications will become apparent to those skilled in the art from a consideration of this disclosure or practice of the development disclosed herein. Consequently, it is not intended that this development be limited to the specific embodiments disclosed herein, but that it covers all modifications and alternatives coming within the true scope and spirit of the development as embodied in the attached claims.

While the above detailed description has shown, described, and pointed out novel features of the improvements as applied to various embodiments, it will be understood that various omissions, substitutions, and changes in the form and details of the device or process illustrated may be made by those skilled in the art without departing from the spirit of the invention. As will be recognized, the present invention may be embodied within a form that does not provide all the features and benefits set forth herein, as some features may be used or practiced separately from others. The scope of the invention is indicated by the appended claims rather than by the foregoing description. All changes which come within the meaning and range of equivalency of the claims are to be embraced within their scope.

What is claimed is:

1. A method for delivering nitric oxide in a bath, comprising:
providing a tub;
filling the tub with approximately one liter of water;
providing a pod, wherein the pod comprises an envelope containing reactants, and the reactants are comprised of a nitrite powder and an acidic powder;
placing the pod in the water;
initiating production of nitric oxide; and
soaking a portion of a user's skin in the water during the production of nitric oxide.

2. The method of claim 1, wherein the reactants are further comprised of a sodium bicarbonate powder.

3. The method of claim 2, wherein the reactants are further comprised of a sodium lauryl sulfoacetate powder.

4. The method of claim 1, wherein the acidic powder comprises one or more of ascorbic acid powder and citric acid powder.

5. The method of claim 3, further comprising:
generating a bubble layer after the initiating the production of nitric oxide.

6. The method of claim 1, wherein the pod is a sphere-shaped pod.

7. The method of claim 1, further comprising:
placing, after placing the pod, a vented tray in the tub so that the portion of the user's skin can be positioned above the pod during the production of nitric oxide without the portion of the user's skin contacting the pod.

8. The method of claim 1, wherein the water is a saline solution.

9. The method of claim 2, the reactants are further comprised of a coco betaine powder.

10. The method of claim 2, the reactants are further comprised of a cocamidopropyl betaine powder.

11. A method for delivering nitric oxide in a bath, comprising:
providing a tub;
filling the tub with approximately one liter of water;
providing a pod, wherein the pod comprises an envelope containing reactants, and the envelope is comprised of polyvinyl alcohol, and the reactants are comprised of a nitrite powder and an acidic powder;
placing the pod in the water;
initiating production of nitric oxide; and
soaking a portion of a user's skin in the water during the production of nitric oxide.

12. The method of claim 11, wherein the reactants further comprise a sodium lauryl sulfoacetate powder.

13. The method of claim 12, further comprising:
generating a bubble layer after the initiating the production of nitric oxide.

14. The method of claim 13, wherein the pod is a sphere-shaped pod.

15. The method of claim 11, wherein the production of nitric oxide comprises the production of at least 20 micromoles of nitric oxide.

16. A method for delivering nitric oxide in a bath, comprising:
providing a tub;
filling the tub with approximately one liter of water;
providing a tablet, wherein the tablet has a mass of approximately 3 g and comprises a nitrite compound, an acidic compound, a surfactant, sodium bicarbonate, Epsom salts, olive oil, and corn starch;
placing the tablet in the water;
initiating production of nitric oxide;
generating a bubble layer that can trap nitric oxide as the nitric oxide leaves the water; and
soaking a portion of a user's skin in the water during the production of nitric oxide.

17. The method of claim 16, wherein the nitrite compound is sodium nitrite and the acidic compound is citric acid.

18. The method of claim 17, wherein the amount of nitric oxide produced is at least 1 micromole of nitric oxide.

19. The method of claim 17, wherein the tablet is sphere-shaped.

20. The method of claim 17, further comprising:
placing, after placing the tablet, a vented tray in the tub so that the portion of the user's skin can be positioned above the tablet during the production of nitric oxide without the portion of the user's skin contacting the tablet.
